(19) United States
(12) Patent Application Publication
(10) Pub. No.: US 2009/0031871 A1
(43) Pub. Date: Feb. 5, 2009

(54) Dual cutting element tool for debulking bone

(76) Inventor: Hugues F. Malandain, Mountain View, CA (US)

Correspondence Address: HAYNES AND BOONE, LLP;IP Section, 2323 Victory Avenue, Suite 700, Dallas, TX, 75219, US

(21) Appl. No.: 11/448,976

(22) Filed: Jun. 8, 2006

(51) Int. Cl. B26D 3/00; A61B 17/32

(52) U.S. Cl. 83/53; 606/79

(57) ABSTRACT

Apparatus and methods for removing selected tissue are described herein. In one embodiment, an apparatus includes a first cutting element including a distal end and a cutting edge at the distal end and a second cutting element including a distal end and a cutting edge at the distal end of the second cutting element. The second cutting element is positioned relative to the first cutting element such that the cutting edge of the first cutting element and the cutting edge of the second cutting element cooperatively produce a shear force on a region of an object when the first cutting element moves in a first direction and the second cutting element moves in a second direction, different from the first direction. The second cutting element defines a conduit configured to convey a fluid from the region of the object.

### BACKGROUND

The invention relates generally to a medical device, and more particularly to an apparatus for removing tissue within a body of a patient.

As a part of various medical procedures, it is often necessary to remove selected tissue from a patient's body. Such tissue removal can be necessary, for example, to reduce pain and/or complications associated with a bone tumor, to define a path within a structure to assist in the surgical repair of the structure and/or to remove scar tissue. Some known devices for removing tissue often include a single cutting element, which can, at times, be inefficient. Other known devices for removing bone often require separate apparatuses for supplying an irrigating fluid to the region of tissue being cut and/or for removing the irrigation fluid and tissue particles from the body.

Thus, a need exists for a medical device that more efficiently removes selected tissue from the body of a patient.

### SUMMARY

Apparatuses and methods for removing body tissue are described herein. In one embodiment, an apparatus includes a first cutting element including a distal end and a cutting edge at the distal end and a second cutting element including a distal end and a cutting edge at the distal end of the second cutting element. The second cutting element is positioned relative to the first cutting element such that the cutting edge of the first cutting element and the cutting edge of the second cutting element cooperatively produce a shear force on a region of an object when the first cutting element moves in a first direction and the second cutting element moves in a second direction, different from the first direction. The second cutting element defines a conduit configured to convey a fluid from the region of the object.

### BRIEF DESCRIPTION OF THE DRAWINGS

FIG. 1 is a schematic illustrating a medical device according to an embodiment of the invention.

FIG. 2A is a perspective view of a medical device according to an embodiment of the invention.

FIG. 2B is a cross-sectional front view of a portion of the medical device illustrated in FIG. 2A.

FIG. 3 is a cross-sectional front view of a portion of a medical device according to an embodiment of the invention.

FIG. 4 is a cross-sectional front view of a portion of a medical device according to an embodiment of the invention.

FIG. 5 is a cross-sectional front view of a portion of a medical device according to an embodiment of the invention.

FIG. 6 is a perspective view of a portion of a medical device according to an embodiment of the invention.

FIG. 7 is a flow chart illustrating a method for removing tissue from a body of a patient according to an embodiment of the invention.

### DETAILED DESCRIPTION

In some embodiments, the apparatus includes a first cutting element having a distal end and a cutting edge at the distal end, and a second cutting element including a distal end and a cutting edge at the distal end of the second cutting element. The second cutting element is positioned relative to the first cutting element such that the cutting edge of the first cutting element and the cutting edge of the second cutting element cooperatively produce a shear force on a region of an object when the first cutting element moves in a first direction and the second cutting element moves in a second direction different from the first direction. In some embodiments, for example, the first direction is a rotational direction about an axis of the first cutting element, and the second direction is a rotational direction about an axis of the second cutting element. The second cutting element defines a conduit configured to convey a fluid from the region of the object.

In some embodiments, an apparatus includes a first cutting element and a second cutting element. The first cutting element includes a cutting edge and a side wall having an inner surface and an outer surface, the inner surface defining a conduit configured to convey a fluid, the outer surface defining a flute. The second cutting element is disposed about the first cutting element and includes a cutting edge. The first cutting element and the second cutting element are each configured to rotate about their respective longitudinal axes. In some embodiments, for example, the longitudinal axis of the first cutting element is substantially parallel to the longitudinal axis of the second cutting element.

In some embodiments, an apparatus includes a first cutting element and a second cutting element. The first cutting element includes a cutting edge and a side wall having an inner surface and an outer surface, the inner surface defining a conduit configured to convey a fluid, the outer surface defining a flute having a helix angle. The second cutting element is disposed about the first cutting element and includes a cutting edge and a side wall having an inner surface and an outer surface, the inner surface of the second cutting element defining a flute having a helix angle. The helix angle of the first cutting element is in an opposite direction from the helix angle of the second cutting element. The first cutting element and the second cutting element are each configured to rotate in a direction about their respective longitudinal axes.

In some embodiments, an apparatus includes a housing, a first cutting element and a second cutting element. The housing defines a conduit configured to convey a fluid from a region of an object. The first cutting element, a portion of which is disposed within the housing, includes a distal end and a cutting edge at its distal end. The second cutting element, a portion of which is disposed within the housing, includes a distal end and a cutting edge at its distal end. The cutting edge of the first cutting element and the cutting edge of the second cutting element are collectively configured to shear a region of the object when the first cutting element moves in a first direction and the second cutting element moves in a second direction different than the first direction.

In some embodiments, an apparatus includes a first element and a second element. The first element is configured to define a first portion of an access path within a body and to cut a region of an object in a first direction. The first element defines a lumen in fluid communication with the region of the object. The second element is configured to define a second portion of the access path within the body and to cut the region of the object in a second direction different from the first direction.

In some embodiments, an apparatus includes a first cutting element, a second cutting element and a conduit. The first cutting element and the second cutting element each include a distal end and a cutting edge at their respective distal ends. The second cutting element is positioned relative to the first cutting element such that the cutting edge of the first cutting element and the cutting edge of the second cutting element cooperatively produce a shear force on a region of an object when the first cutting element moves in a first direction and the second cutting element moves in a second direction different from the first direction. The conduit has a first opening and a second opening disposed apart from the first opening, the conduit being configured such that the first opening can be disposed proximate to the region of the object.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of the cutting element first inserted inside the patient's body would be the distal end of the cutting element, while the end of the cutting element to last enter the patient's body would be the proximal end of the cutting element.

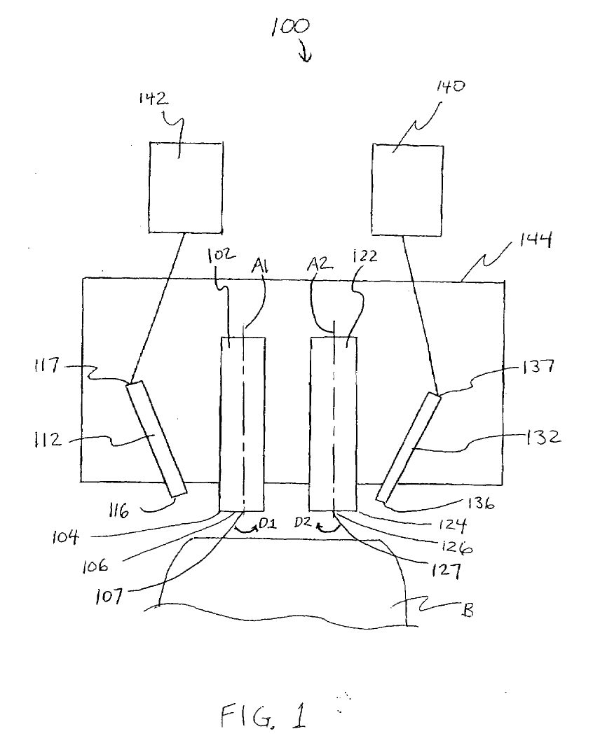

FIG. 1 is a schematic illustration of a medical device 100 according to an embodiment of the invention. The medical device 100 includes a first cutting element 102 and a second cutting element 122. The first cutting element 102, which is configured to move in a direction D1, includes a distal end 104 and a cutting portion 106 located at the distal end 104. Similarly, the second cutting element 122, which is configured to move in a direction D2, includes a distal end 124 and a cutting portion 126 located at the distal end 124. In some embodiments, a portion of the first cutting element 102 and a portion of the second cutting element 122 are disposed within a housing 144. The second cutting element 122 is positioned relative to the first cutting element 102 such that the cutting portion 106 of the first cutting element 102 and the cutting portion 126 of the second cutting element 122 cooperatively produce a shear force on an object B when the first cutting element 102 moves in direction D1 and the second cutting element 122 moves in direction D2. In some embodiments, direction D1 can be different from direction D2.

In some embodiments, the medical device 100 includes a first conduit 112 and a second conduit 132. The first conduit 112 includes a first opening 116 and a second opening 117 disposed apart from the first opening 116. As illustrated in FIG. 1, the first opening 116 can be disposed proximate to or adjacent the region of object B in which the shear force is produced. The second opening 117 is in fluid communication with a fluid supply source 142. The fluid supply source 142 can be, for example, a pump configured to supply a pressurized irrigation fluid, such as saline solution, a therapeutic gel and the like, to object B via the first conduit 112 and through the first opening 116. In this manner, the temperature of object B can be maintained or modified as desired during the tissue cutting procedure. Additionally, the irrigation fluid can serve to flush out tissue particles once they have been removed from object B. The irrigation fluid can also facilitate the transport of removed tissue particles from object B through the second conduit 132, as described below.

Similarly the second conduit 132 includes a first opening 136 and a second opening 137 disposed apart from the first opening 136. The first opening 136 can be disposed proximate to or adjacent the region of object B in which the shear force is produced. The second opening 137 is in fluid communication with a suction source 140. The suction source 140 can be, for example, a suction pump configured to generate a negative pressure to remove the irrigation fluid and tissue particles from the object B via the second conduit 132.

The first cutting portion 106 and the second cutting portion 126 each include a cutting edge 107 and 127, respectively. The cutting edges 107, 127 can be configured in any manner suitable for cutting tissue, such as bone, bone tumor and/or cartilage. In some embodiments, for example, the cutting portions 106, 126 each can include a cutting edge, similar to the type of cutting edge found on a drill bit or a single-point cutter. In other embodiments, the cutting portions 106, 126 each can include a series of cutting edges, such as, for example, a saw-tooth configuration. In yet other embodiments, the cutting portions 106, 126 each can include an abrasive cutting edge, similar to the type of cutting edge used in grinding operations. The cutting edges 107 and 127 can have either the same or different configurations. In some embodiments, for example, the first cutting portion 106 is configured as a drill bit having a single-point cutting edge 107, while the second cutting portion 126 is configured as a saw-tooth cutter having a series of cutting edges 127. Conversely, in other embodiments, the first cutting portion 106 and the second cutting portion 126 can each include a single-point cutting edge.

Similarly, the cutting edge 107 and the cutting edge 127 can have any geometry suitable for cutting tissue. For example, in some embodiments, either of the cutting edges 107, 127 can have a positive rake angle. Conversely, in other embodiments, either of the cutting edges 107, 127 can have a negative rake angle.

In some embodiments, the cutting elements 102, 122 are monolithically formed with their respective cutting portions 106, 126. In other embodiments, the cutting portions 106, 126 are separate components coupled to their respective cutting elements 102, 122. In some embodiments, for example, the cutting portions 106, 126 can be fabricated from a material formulated to provide the requisite material properties for optimal tissue cutting, as will be discussed herein, whereas the other portions of the cutting elements 102, 122 can be fabricated from a different material. In other embodiments, the cutting portions 106, 126 can be removably coupled to their respective cutting elements 102, 122. In this manner, the medical device 100 can be selectively configured to cut a specific type of tissue. Moreover, such a configuration allows the cutting portions 106, 126 to be replaced when the cutting edges 107, 127 become worn and/or damaged.

The cutting portions 106, 126 can be fabricated from any material having suitable properties for cutting tissue within a patient's body. Such properties include, for example, biocompatibility, high hardness and/or high strength. For example, in some embodiments the cutting portions 106, 126 can be fabricated from a material, such as stainless steel selected from the 400 series, having a hardness sufficient to cut bone. In other embodiments, such as, for example, those in which the medical device is configured to cut cartilage, the cutting portions 106, 126 can be fabricated from a material having a relatively lower hardness. In yet other embodiments, the material from which the cutting portions are fabricated is not limited to stainless steel alloys. For example, in some embodiments, the cutting portions can be fabricated from other metallic alloys, ceramic compositions, carbide, or any other material suitable for fabricating a cutting edge.

As described above, the first cutting element 102 is configured to move in a direction D1, and the second cutting element 122 is configured to move in a direction D2. The movement of the cutting elements 102, 122 can be a rotational movement and/or a translational movement. In some embodiments, each of the cutting elements 102, 122 can be configured to rotate about their respective longitudinal axes A1, A2. In such embodiments, for example, the rotational direction D1′ of the first cutting element 102 can be clockwise about longitudinal axis A1, while the rotational direction D2′ of the second cutting element 122 can be counter-clockwise about longitudinal axis A2. In other embodiments, the cutting elements 102, 122 can be configured to translate reciprocally in a direction D1″, D2″ normal to their respective longitudinal axes A1, A2. In yet other embodiments, the cutting elements 102, 122 can be configured to translate reciprocally in a direction D1″, D2″ parallel to their respective longitudinal axes A1, A2. In still other embodiments, each of the first and second cutting elements 102, 122 are configured to both translate and rotate.

Similarly, each of the first and second cutting elements 102, 122 can configured to move selectively in multiple different directions. In some embodiments, for example, the first cutting element 102 and/or the second cutting element 122 can be configured to rotate about longitudinal axes A1 and A2, respectively, in either a clockwise and a counter-clockwise direction. In this manner, a user can selectively determine the appropriate direction as circumstances dictate. For example, in some instances, it may be desirable to reverse the direction of rotation of the cutting elements.

Each of the first and second cutting elements 102, 122 can be configured to be selectively moved at different speeds. In some embodiments, for example, the cutting elements 102, 122 can be configured to be actuated at one of any number of distinct speed settings (i.e., slow, medium and fast). In other embodiments, the cutting elements 102, 122 can be configured to be actuated at any speed within a continuum of speeds. In this manner, a user can adjust the speed of the cutting elements to optimize or improve the tissue cutting process.

Each of the first and second cutting elements 102, 122 can be driven by any device (not shown) suitable for use in such applications. In some embodiments, for example, the cutting elements can be driven by a single motor (not shown). Such a motor can be powered electrically, pneumatically and/or hydraulically. In other embodiments, the first cutting element 102 can be driven by a first motor (not shown), while the second cutting element 122 can be driven by a second motor (not shown). In this manner, the first cutting element 102 and the second cutting element 122 can be actuated independently of each other. In yet other embodiments, the cutting elements can be driven by a single motor (not shown) via a clutch mechanism (not shown). In this manner, the cutting elements can be driven at different speeds and in different directions using a single motor.

The first conduit 112 and the second conduit 132 can be any structure suitable for forming a channel (not shown) through which a fluid can be conveyed. In some embodiments, for example, the first conduit 112 and the second conduit 132 can each be a tube having a flexible portion terminating at one end at the first opening 116, 136. In this manner, the location of the first opening 116, 136 can be selectively positioned as required by the circumstances. In other embodiments, the first conduit 112 and the second conduit 132 can be channels defined by the housing 144. In yet other embodiments, the first conduit 112 and the second conduit 132 can be channels defined by the first cutting element 102, the second cutting element 122 and/or a combination of the first cutting element 102 and the second cutting element 122.

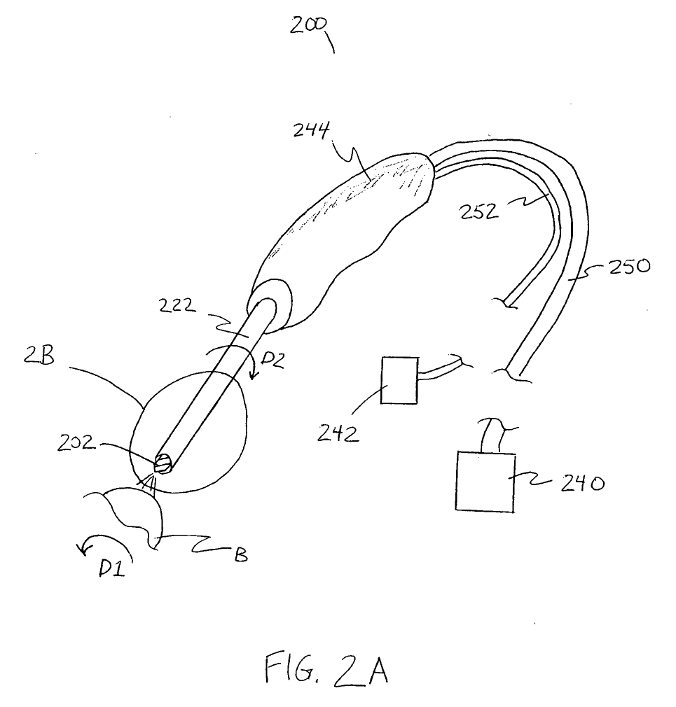

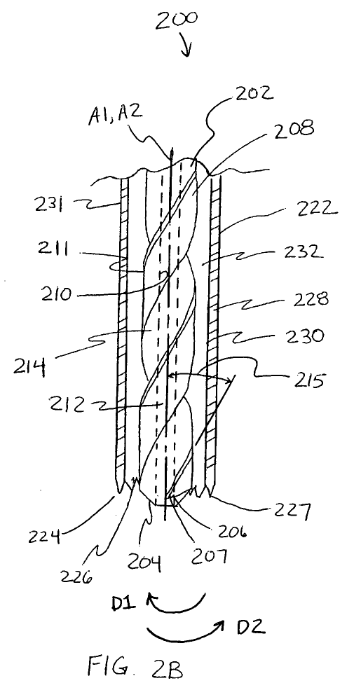

FIGS. 2A and 2B illustrate a medical device 200 according to an embodiment of the invention that includes a first cutting element 202 disposed within a second cutting element 222. As illustrated in FIG. 2A, a portion of each of the first cutting element 202 and the second cutting element 222 are disposed within housing 244. The housing 244 can be configured to allow a user to insert and position the distal end portion of the medical device 200 within a patient. In some embodiments, the housing 244 provides a suitable location for the controls (not shown) to operate the medical device 200.

As illustrated in FIG. 2B, the first cutting element 202 includes a distal end 204 and a cutting portion 206 located at the distal end 204. The cutting portion 206 includes a cutting edge 207. The first cutting element 202 also includes a side wall 208 having an inner surface 210 and an outer surface 211. The inner surface 210 defines a first conduit 212, through which an irrigating fluid can be conveyed. The first conduit 212 is coupled to and in fluid communication with a fluid supply source 242 via connection tube 252 (see FIG. 2A). The outer surface 211 defines a flute 214 having a helix angle 215 having a direction consistent with the intended direction of rotation D1 of the first cutting element 202 when operating to cut tissue. As described in more detail herein, the flute 214 is configured to convey or aid in the conveyance of the irrigation fluid, bodily fluid and/or any tissue particles away from object B when the first cutting element 202 rotates in its intended direction of rotation. A helix angle consistent with the intended direction of rotation, therefore, is one which aids in the transportation of the irrigation fluid, bodily fluid and/or tissue particles away from object B.

The second cutting element 222 includes a distal end 224 and a cutting portion 226 located at the distal end 224. In the illustrated embodiment, the cutting portion 226 includes multiple cutting edges 227 in a saw-tooth configuration. The second cutting element 222 also includes a side wall 228 having an inner surface 230 and an outer surface 231. The first cutting element 202 is disposed within the second cutting element 222 such that the outer surface 211 of the side wall 208 of the first cutting element 202 and the inner surface 230 of the side wall 228 of the second cutting element 222 define a second conduit 232. The second conduit 222 is coupled to and in fluid communication with a suction source 240 via connection tube 250 (see FIG. 2A).

As illustrated, the first cutting element 202 is disposed within and is concentric with the second cutting element 222. In this manner, the cutting edge 207 of the first cutting element 202 and the cutting edges 227 of the second cutting element 222 are configured to produce cooperatively a shear force on an object B when the first cutting element 202 rotates in direction D1 about its longitudinal axis A1 and the second cutting element 222 rotates in direction D2 about its longitudinal axis A2.

In operation, as the first cutting element 202 rotates in direction D1, the flute 214 is configured to convey the irrigation fluid, bodily fluid and/or any tissue particles away from object B via the second conduit 232 (i.e., from the cutting edge or the distal end of the device to the proximal end of the device). In some embodiments, the flute 214 is configured to displace the irrigation fluid, bodily fluid and/or any tissue particles by forcing them via the angled structures of the flute 214 in an upward direction as the cutting element 202 rotates in direction D1. In other embodiments, the radial spacing between the outer surface 211 of the side wall 208 and the inner surface 230 of the side wall 228 is such that the rotation of the first and second cutting elements 202, 232 creates an area of reduced pressure or suction within the second conduit 232, which acts to improve the transport of the irrigation fluid, bodily fluid and/or any tissue particles away from object B. Such an area of reduced pressure need not have a pressure below atmospheric pressure, but need only have a pressure that is relatively lower than the pressure in surrounding areas.

Although the outer surface 211 of the side wall 208 is illustrated as defining a single flute 214, in some embodiments, the outer surface of the side wall defines multiple flutes. In some embodiments, for example, the outer surface of the side wall can define four flutes, thereby allowing the irrigation fluid, bodily fluid and/or any tissue particles to be removed more quickly. Moreover, the flute 214 can be a groove or channel of any shape suitable for transporting the irrigation fluid, bodily fluid and/or any tissue particles away from object B. For example, in some embodiments, the flute can have a relatively circular cross-sectional shape. In other embodiments, the flute can have a discontinuous cross-sectional shape, such as for example, a flattened portion and a curved portion. Similarly, the helix angle 215 of the flute 214 can be any suitable value. In some embodiments, for example, the flute 214 can have a high helix angle (i.e., a helix angle greater than about 30 degrees) to improve the transport of the irrigation fluid, bodily fluid and/or any tissue particles.

In some embodiments, the medical device 200 is inserted percutaneously (i.e., through an opening in the skin) and/or in a minimally-invasive manner. For example, in some embodiments, a trocar enclosed within a cannula (not shown) can be used to define an access passageway (not shown) for the medical device 200. The trocar can be a separate device that is removed upon defining the access passageway. In other embodiments, the distal end of the first cutting element and/or the second cutting element are configured to define the access passageway, thereby eliminating the need for a separate device to define the access passageway.

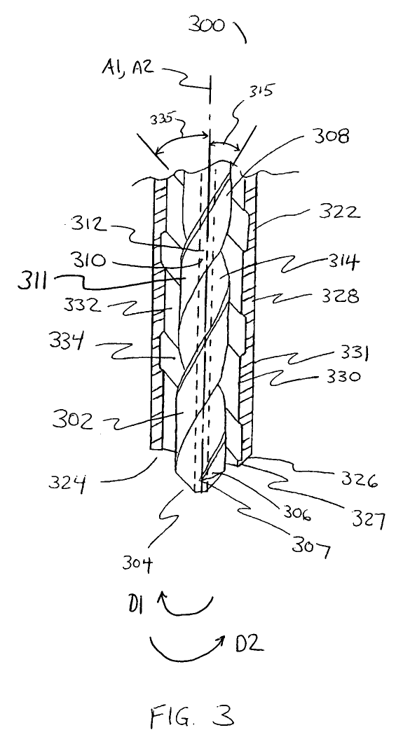

FIG. 3 is a cross-sectional front view of a portion of a medical device 300 according to an embodiment of the invention that includes a first cutting element 302 disposed within a second cutting element 322. As illustrated, the first cutting element 302 includes a distal end 304 and a cutting portion 306 located at the distal end 304. The cutting portion 306 includes a cutting edge 307. The first cutting element 302 also includes a side wall 308 having an inner surface 310 that defines a first conduit 312, and an outer surface 311 that defines a flute 314 having a helix angle 315 in a direction consistent with the intended direction of rotation D1 of the first cutting element 302 when operating to cut tissue.

The second cutting element 322 includes a distal end 324 and a cutting portion 326 located at the distal end 324. Unlike the second cutting element 222 shown and described above as including a multiple cutting edges in a saw-tooth configuration, the cutting portion 326 includes a single-point style cutting edge 327. The second cutting element 322 also includes a side wall 328 having an inner surface 330 and an outer surface 331. The inner surface 330 of the side wall 328 defines an internal flute 334 having a helix angle 335 in a direction consistent with the intended direction of rotation D2 of the second cutting element 322 when operating to cut tissue. In some embodiments, the direction of the flute 314 is opposite the direction of the internal flute 334. The first cutting element 302 is disposed within the second cutting element 322 such that the outer surface 311 of the side wall 308 of the first cutting element 302 and the inner surface 330 of the side wall 328 of the second cutting element 322 define a second conduit 332.

As illustrated, the first cutting element 302 is disposed within and concentric with the second cutting element 322. In this manner, the cutting edge 307 of the first cutting element 302 and the cutting edge 327 of the second cutting element 322 can collectively produce a shear force on a region of an object (not shown) when the first cutting element 302 rotates in direction D1 about its longitudinal axis A1 and the second cutting element 322 rotates in direction D2 about its longitudinal axis A2.

In operation, as the first cutting element 302 rotates in direction D1 and the second cutting element 322 rotates in direction D2, the cutting edge 307 and the cutting edge 327 collectively produce a shear force on a region of an object (not shown). Additionally, the second conduit 332 via flutes 314, 334 transports the irrigation fluid, bodily fluid and/or any tissue particles away from the object via the second conduit 332, as the first cutting element 302 and the second cutting element 322 rotate in opposite directions. As described above, in some embodiments, the outer surface 311 of the side wall 308 can define multiple flutes. Similarly, the inner surface 330 of the side wall 328 can define multiple internal flutes.

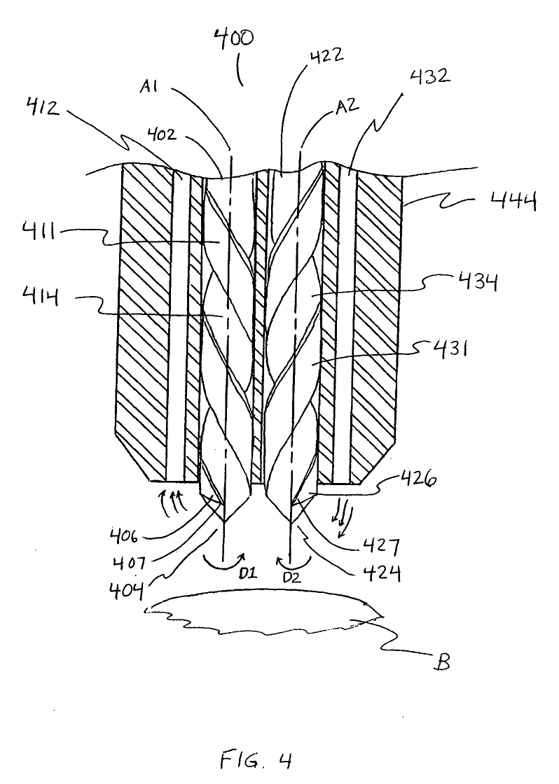

FIG. 4 is a cross-sectional front view of a portion of a medical device 400 according to an embodiment of the invention that includes a first cutting element 402 and a second cutting element 422. A portion of each of the first cutting element 402 and the second cutting element 422 is disposed within a housing 444. The first cutting element 402 includes a distal end 404 and a cutting portion 406 located at the distal end 404. The cutting portion 406 includes a cutting edge 407. The first cutting element 402 also includes an outer surface 411 that defines a flute 414, as described above. Similarly, the second cutting element 422 includes a distal end 424 and a cutting portion 426 located at the distal end 424. The cutting portion 426 includes a cutting edge 427. The second cutting element 422 also includes an outer surface 431 that defines a flute 434, as described above.

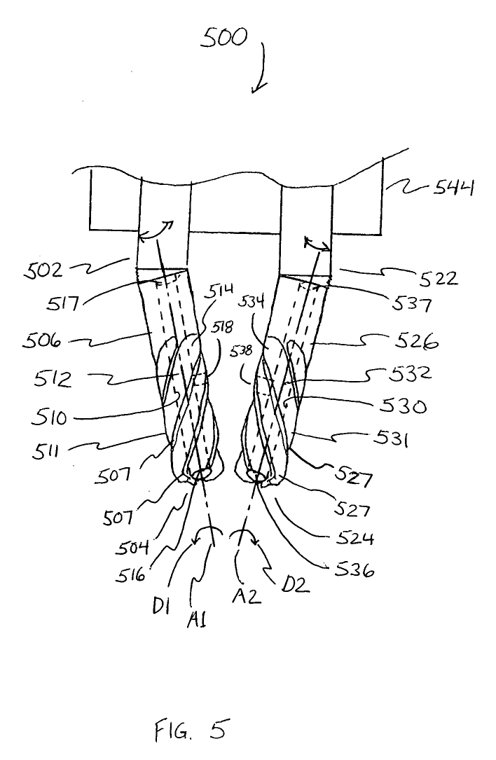

The first cutting element 402 is disposed parallel to and adjacent the second cutting element 422 such that the cutting edge 407 of the first cutting element 402 and the cutting edge 427 of the second cutting element 422 collectively produce a shear force on a region of object B when the first cutting element 402 rotates in direction D1 about its longitudinal axis A1 and the second cutting element 422 rotates in direction D2 about its longitudinal axis A2. Although the longitudinal axes A1 and A2 are shown as being parallel, in some embodiments, the longitudinal axes of the cutting elements are not parallel (see FIG. 5, discussed in more detail herein).

In the illustrated embodiment, the housing 444 defines a first conduit 412 and a second conduit 432. The first conduit 412 can be coupled to and in fluid communication with a suction source, as previously described. In this manner, irrigation fluid, bodily fluid and/or cut tissue particles can be transported away from the object, as indicated by the arrows. Similarly, the second conduit 432 can be coupled to and in fluid communication with a fluid supply source, as previously described. In this manner, irrigation fluid can be supplied to the object, as indicated by the arrows.

FIG. 5 is a front view of a portion of a medical device 500 according to an embodiment of the invention that includes a first cutting element 502 and a second cutting element 522. A portion of each of the first cutting element 502 and the second cutting element 522 is disposed within a housing 544. The first cutting element 502 includes a cutting portion 506 having distal end 504, an outer surface 511 and an inner surface 510 that defines a conduit 512. The cutting portion 506 includes multiple cutting edges 507 disposed along the distal end 504 and the outer surface 511 of the cutting portion 506. In this manner, the first cutting element 502 is configured to cut at both the distal end 504 and along the periphery of the cutting portion 506, similar to an end mill type cutter. The outer surface 511 also defines multiple flutes 514, as described above.

The conduit 512 includes a first opening 516, a second opening 517 and a third opening 518. The second opening 517 is disposed apart from the first opening 516; the third opening 518 is disposed between the first opening 516 and the second opening 517. The second opening 517 can be in fluid communication with a fluid supply source (not shown), as discussed above. The first opening 516 and the third opening 518 are configured to be disposed proximate to an object (not shown) being cut. In this manner, irrigation fluid can be supplied to all regions of the object being cut (i.e., the region adjacent the end portion 504 and the region adjacent the outer surface 511).

Similarly, the second cutting element 522 includes a cutting portion 526 having distal end 524, an outer surface 531 and an inner surface 530 that defines a conduit 532. The cutting portion 526 includes multiple cutting edges 527 disposed along both the distal end 524 and the outer surface 531 of the cutting portion 526, as described above. The outer surface 531 also defines multiple flutes 534, as described above. As described above, the conduit 532 includes a first opening 536, a second opening 537 and a third opening 538. The second opening 537 can be in fluid communication with a suction source (not shown), as discussed above. The first opening 536 and the third opening 538 are configured to be disposed proximate to the object (not shown) being cut. In this manner, irrigation fluid, bodily fluid and/or cut tissue particles can be transported away from the regions of the object being cut.

As illustrated, the first cutting element 502 is disposed adjacent the second cutting element 522 such that a longitudinal axis A1 of the first cutting element 502 is not parallel to a longitudinal axis A2 of the second cutting element 522. In this manner, the cutting edges 507 and the cutting edges 527 are configured collectively to produce a shear force on a region of the object at both the distal end and along a portion of the periphery of each cutting element 502, 522 when the first cutting element 502 rotates in direction D1 about its longitudinal axis A1 and the second cutting element 522 rotates in direction D2 about its longitudinal axis A2.

In some embodiments, the cutting elements can be coupled to a drive device, for example, a motor of the type discussed above (not shown), via a coupling member (not shown) configured to transmit rotary motion from the drive device to the cutting elements while changing the axis of rotation. Such a coupling member can include, for example, a universal-joint, a flexible drive wire and the like. In other embodiments, for example, the orientation of each cutting element can be adjusted thereby allowing the relative orientation of the longitudinal axes of the cutting elements to be adjusted.

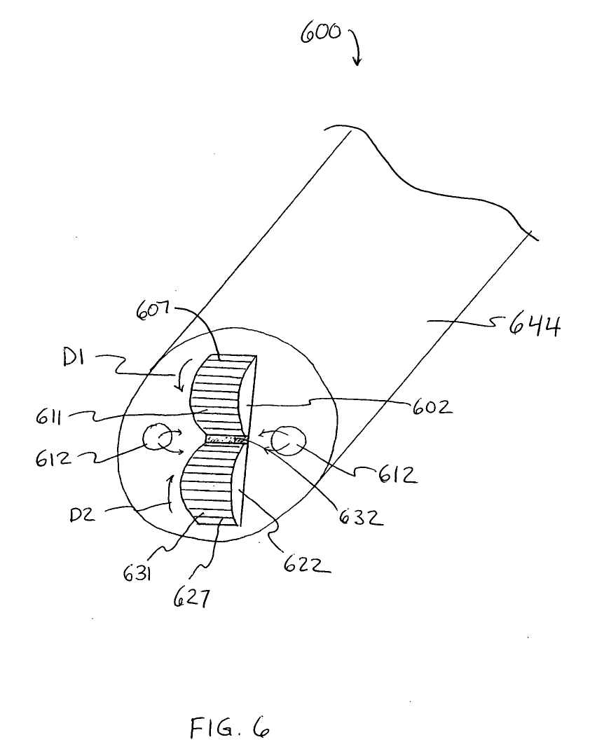

FIG. 6 is a perspective view of a portion of a medical device 600 according to an embodiment of the invention that includes a first cutting element 602 and a second cutting element 622. A portion of each of the first cutting element 602 and the second cutting element 622 is disposed within a housing 644. The first cutting element 602 is cylindrically shaped and includes an outer surface 611 that defines a series of cutting edges 607. Similarly, the second cutting element 622 is cylindrically shaped and includes an outer surface 631 that defines a series of cutting edges 627.

The first cutting element 602 is disposed adjacent the second cutting element 622 such that the cutting edges 607 of the first cutting element 602 and the cutting edges 627 of the second cutting element 622 collectively produce a shear force on a region of an object (not shown) when the first cutting element 602 rotates in direction D1 and the second cutting element 622 rotates in direction D2.

In the illustrated embodiment, the housing 644 defines two fluid supply conduits 612 and a fluid removal conduit 632 disposed between the first cutting element 602 and the second cutting element 622. The fluid supply conduits 612 can coupled to and in fluid communication with a fluid supply source, as previously described. In this manner, irrigation fluid can be supplied to the object, as indicated by the arrows. Similarly, the removal conduit 632 can be coupled to and in fluid communication with a suction source, as previously described.

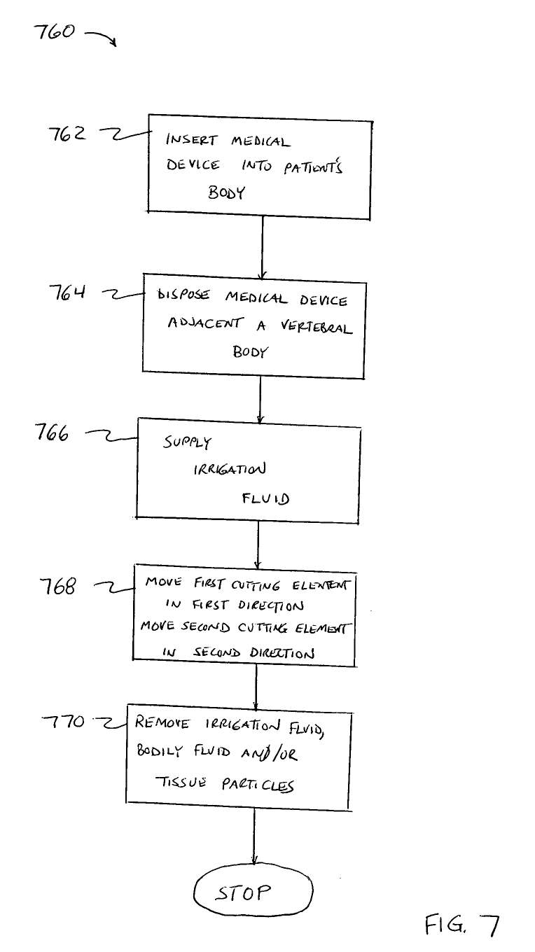

FIG. 7 is a flow chart illustrating a method 760 for removing tissue from a body of a patient according to an embodiment of the invention. The illustrated method includes inserting (at 762) a medical device having a first cutting element and a second cutting element, of the type described above, into the body of a patient. As described above, in some embodiments, the medical device can be inserted percutaneously and/or in a minimally-invasive manner. In some embodiments, the medical device is inserted into an access passageway previously defined by a separate device, such as a trocar. In other embodiments, the first cutting element and/or the second cutting element are configured to define the access passageway, thereby eliminating the need for a separate device to define the access passageway.

At 764, the medical device is disposed adjacent an object (e.g., a vertebral body) such that that a cutting edge of the first cutting element and a cutting edge of the second cutting element are positioned adjacent a region of the bodily tissue within the object to be removed.

At 766, an irrigation fluid is supplied to the region of the object to be removed. The irrigation fluid can be supplied, for example, via a conduit included in the medical device, as described above. In some embodiments, the step of supplying an irrigation fluid is not necessary.

The illustrated method then includes moving the first cutting element in a first direction and the second cutting element in a second direction different from the first direction at 768. In this manner, the cutting edges of the first and the second cutting elements collectively produce a shear force on the region of the object when the first cutting element moves in the first direction and the second cutting element moves in the second direction. In some embodiments, the first cutting element can be moved independently from and at different speeds than the second cutting element. In other embodiments, the direction of motion can be changed, as described above.

At 770, the irrigation fluid, any bodily fluid and/or any cut tissue particles can be transported away from the region of the object. The irrigation fluid, bodily fluid and/or any cut tissue particles can be removed, for example, by suction via a conduit included in the medical device, as described above. In some embodiments, the step of removing the irrigation fluid, any bodily fluid and/or any cut tissue particles is not necessary for the successful operation of the method.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, although the many of the cutting elements have been shown and described as having one or more cutting edges at a distal end thereof, in some embodiments, one or more cutting edges can be disposed on a peripheral portion of the cutting element.

Although the medical devices have been described as including two cutting elements, in some embodiments, a medical device can have three or more cutting elements. In one variation, for example, a medical device can include three cutting elements, the first of which is disposed within and concentric with the second, and the second of which is disposed within and concentric with the third. In this manner, three distinct styles of cutting edges can be configured cooperatively to produce a shear force on a region of an object.

Although the medical device have been described primarily as being configured to remove a tumor in a vertebral body, in some embodiments, a medical device can be configured to remove other types of tissue from other objects within a patient's body. For example, in some embodiments, a medical device can be configured to remove cartilage from a joint. Moreover, in some embodiments, a medical device can be configured to produce a shear force on only a region of an object, such as a vertebral body, whereas in other embodiments, a medical device can be configured to produce a shear force on an entire object within a patient's body.

What is claimed is:

1. An apparatus, comprising:
a first cutting element including a distal end and a cutting edge at the distal end; and
a second cutting element including a distal end and a cutting edge at the distal end of the second cutting element, the second cutting element being positioned relative to the first cutting element such that the cutting edge of the first cutting element and the cutting edge of the second cutting element cooperatively produce a shear force on a region of an object when the first cutting element moves in a first direction and the second cutting element moves in a second direction different from the first direction,
the second cutting element defining a conduit configured to convey a fluid.

2. The apparatus of claim 1, wherein:
the conduit is configured to convey the fluid away from the region of the object; and
the first cutting element defines a conduit configured to convey an irrigation fluid to the region of the object.

3. The apparatus of claim 1, further comprising a suction source in fluid communication with the conduit.

4. The apparatus of claim 1, wherein the first cutting element defines a conduit configured to convey an irrigation fluid to the region of the object, the apparatus further comprising:
a suction source in fluid communication with the conduit defined by the second cutting element; and
a fluid source in fluid communication with the conduit defined by the first cutting element.

5. The apparatus of claim 1, wherein:
the first direction is a rotational direction about an axis of the first cutting element; and
the second direction is a rotational direction about an axis of the second cutting element.

6. The apparatus of claim 1, wherein:
the first direction is a rotational direction about an axis of the first cutting element;
the second direction is a rotational direction about an axis of the second cutting element; and
the axis of the second cutting element is substantially parallel to the axis of the first cutting element.

7. The apparatus of claim 1, wherein:
the first direction is a rotational direction about an axis of the first cutting element;
the second direction is a rotational direction about an axis of the second cutting element; and
at least one of the first cutting element or the second cutting element is selectively adjustable between a first configuration, in which its axis is in a first position, and a second configuration, in which its axis is in a second position, different from the first position.

8. The apparatus of claim 1, wherein the first cutting element is disposed within and is substantially concentric with the second cutting element.

9. The apparatus of claim 1, wherein the cutting edge of the first cutting element and the cutting edge of the second cutting element are configured to cut at least one of a bone tissue, a bone tumor, or a cartilage.

10. The apparatus of claim 1, wherein at least one of the cutting edge of the first cutting element or the cutting edge of the second cutting element includes a material different than a material included within a remaining portion of the respective cutting element.

11. The apparatus of claim 1, wherein at least one of the cutting edge of the first cutting element or the cutting edge of the second cutting element is removably coupled to the first cutting element and the second cutting element, respectively.

12. The apparatus of claim 1, wherein the first cutting element is configured to be actuated independently from the second cutting element.

13. The apparatus of claim 1, wherein the conduit defined by the second cutting element is configured to convey at least one of a liquid, a slurry, or a colloidal mixture.

14. A method of cutting the region of the object in a patient's body, comprising:
inserting the apparatus according to claim 1 into the body such that the cutting edge of the first cutting element and the cutting edge of the second cutting element are positioned adjacent the region of the object; and
moving the first cutting element in the first direction and the second cutting element in the second direction.

15. An apparatus, comprising:
a first cutting element configured to rotate in a direction about a longitudinal axis of the first cutting element, the first cutting element including a cutting edge and a side wall having an outer surface, the outer surface defining a flute; and
a second cutting element disposed about the first cutting element, the second cutting element being configured to rotate in a direction about a longitudinal axis of the second cutting element, the second cutting element including a cutting edge.

16. The apparatus of claim 15, wherein the direction of the second cutting element is opposite from the direction of the first cutting element.

17. The apparatus of claim 15, wherein the longitudinal axis of the first cutting element is substantially parallel to the longitudinal axis of the second cutting element.

18. The apparatus of claim 15, wherein the first cutting element is substantially concentric with the second cutting element.

19. The apparatus of claim 15, wherein the first cutting element defines a conduit configured to convey a fluid.

20. The apparatus of claim 15, wherein:
the first cutting element defines a first conduit;
the second cutting element includes a side wall having an inner surface and an outer surface; and
the outer surface of the side wall of the first cutting element and the inner surface of the side wall of the second cutting element collectively define a second conduit configured to convey the fluid.

21. The apparatus of claim 15, wherein:
the second cutting element includes a side wall having an inner surface and an outer surface;
the outer surface of the side wall of the first cutting element and the inner surface of the side wall of the second cutting element collectively define a conduit configured to convey the fluid; and
the first cutting element and the second cutting element are configured to collectively lower a pressure within the conduit when the first cutting element and the second cutting element rotate.

22. The apparatus of claim 15, wherein:
the second cutting element includes a side wall having an inner surface and an outer surface;
the outer surface of the side wall of the first cutting element and the inner surface of the side wall of the second cutting element collectively define a conduit configured to convey the fluid; and
the first cutting element and the second cutting element are configured to collectively displace the fluid within the conduit away from the cutting edge of the first cutting element when the first cutting element and the second cutting element rotate.

23. The apparatus of claim 15, wherein the second cutting element includes a side wall having an inner surface and an outer surface, the inner surface of the second cutting element defining a flute.

24. The apparatus of claim 15, wherein:
the flute of the first cutting element defines a helix angle;
the second cutting element includes a side wall having an inner surface and an outer surface, the inner surface of the second cutting element defining a flute defining a helix angle; and
the helix angle of the first cutting element is in an opposite direction from the helix angle of the second cutting element.

25. The apparatus of claim 15, wherein the cutting edge of the first cutting element and the cutting edge of the second cutting element are configured to cooperatively produce a shear force on an object when the first cutting element and the second cutting element rotate.

26. A method of cutting an object in a patient's body, comprising:
inserting the apparatus according to claim 15 into the body such that the cutting edge of the first cutting element and the cutting edge of the second cutting element are positioned adjacent the object; and
rotating the first cutting element and the second cutting element.

27. An apparatus, comprising:
a housing defining a conduit configured to convey a fluid from a region of an object;
a first cutting element, at least a portion of which is disposed within the housing, the first cutting element including a distal end and a cutting edge at the distal end; and
a second cutting element, at least a portion of which is disposed within the housing, the second cutting element including a distal end and a cutting edge at the distal end of the second cutting element,
the cutting edge of the first cutting element and the cutting edge of the second cutting element being collectively configured to shear a region of the object when the first cutting element moves in a first direction and the second cutting element moves in a second direction different than the first direction.

28. The apparatus of claim 27, further comprising a suction source in fluid communication with the conduit.

29. The apparatus of claim 27, wherein the housing defines a second conduit configured to convey an irrigation fluid to a region of the object.

30. The apparatus of claim 27, wherein the housing defines a second conduit configured to convey an irrigation fluid to a region of the object, the apparatus further comprising:
a suction source in fluid communication with the first conduit; and
a fluid source in fluid communication with the second conduit.

31. The apparatus of claim 27, wherein:
the first direction is a rotational direction about an axis of the first cutting element;
the second direction is a rotational direction about an axis of the second cutting element; and
the axis of the second cutting element is substantially parallel to the axis of the first cutting element.

32. The apparatus of claim 27, wherein:
the first direction is a rotational direction about an axis of the first cutting element;
the second direction is a rotational direction about an axis of the second cutting element; and
the first cutting element is selectively adjustable between a first configuration, in which its axis is in a first position, and a second configuration, in which its axis is in a second position, different from the first position.

33. The apparatus of claim 27, wherein the first cutting element is disposed within and substantially concentric with the second cutting element.

34. An apparatus, comprising:
a first element configured to define a first portion of an access path within a body and to cut a region of an object in a first direction; and
a second element configured to define a second portion of the access path within the body and to cut the region of the object in a second direction different from the first direction,
the first element defining a lumen in fluid communication with the region of the object.

35. An apparatus, comprising:
a first cutting element including a distal end and a cutting edge at the distal end;
a second cutting element including a distal end and a cutting edge at the distal end of the second cutting element, the second cutting element being positioned relative to the first cutting element such that the cutting edge of the first cutting element and the cutting edge of the second cutting element cooperatively produce a shear force on a region of an object when the first cutting element moves in a first direction and the second cutting element moves in a second direction different from the first direction; and
a conduit having a first opening and a second opening disposed apart from the first opening, the conduit configured such that the first opening can be disposed proximate to the region of the object.

36. The apparatus of claim 35, further comprising a fluid source in fluid communication with the second opening of the conduit.

37. The apparatus of claim 35, wherein the conduit includes any one of a tube, a conduit defined by the first cutting element, a conduit defined by the second cutting element or a conduit defined by a housing in which a portion of either of the first cutting element or the second cutting element is disposed.

38. The apparatus of claim 35, wherein the conduit is a first conduit, the apparatus further comprising a second conduit having a first opening and a second opening disposed apart from the first opening, the second conduit configured such that the first opening of the second conduit can be disposed proximate to the region of the object.

39. The apparatus of claim 35, wherein the conduit is a first conduit, the apparatus further comprising:
a fluid source in fluid communication with the second opening of the first conduit;
a second conduit having a first opening and a second opening disposed apart from the first opening, the second conduit configured such that the first opening of the second conduit can be disposed proximate to the region of the object; and
a suction source in fluid communication with the second opening of the second conduit.

40. A method, comprising:
introducing into a body an apparatus having:
a first cutting element including a distal end and a cutting edge at the distal end; and
a second cutting element including a distal end and a cutting edge at the distal end of the second cutting element, the second cutting element being positioned relative to the first cutting element such that the cutting edge of the first cutting element and the cutting edge of the second cutting element cooperatively produce a shear force on a region of a vertebral body;
disposing the apparatus such that the cutting edge of the first cutting element and the cutting edge of the second cutting element are positioned adjacent the region of the vertebral body; and
moving the first cutting element in a first direction and the second cutting element in a second direction different from the first direction.

41. The method of claim 40, further comprising removing a fluid from an area adjacent the region of the vertebral body.

42. The method of claim 40, further comprising supplying a fluid to an area adjacent the region of the vertebral body.

43. The method of claim 40, wherein the moving the first cutting element is independent from the moving the second cutting element.

44. The method of claim 40, wherein the introducing is done without a trocar.

45. The method of claim 40, wherein the introducing is done percutaneously.
